SEQUENCE LISTING (16 sequences)

SEQ ID NO: 1 - DNA, 1057 bp, Homo sapiens
  CDS (32)..(1012)
atcacaggtgtccctctctgcacctgcgcagatgtgccctggcctgagcgaggccccggaactctacagccggggcttcctgaccatcgagcagatcgcgatgctgccgcctccggccgtcatgaactacatcttcctgctcctctgcctgtgtggcctggtgggcaacgggctggtcctctggtttttcggcttctccatcaagaggaaccccttctccatctacttcctgcacctggccagcgccgatgtgggctacctcttcagcaaggcggtgttctccatcctgaacacggggggcttcctgggcacgtttgccgactacatccgcagcgtgtgccgggtcctggggctctgtatgttccttaccggcgtgagcctcctgccggccgtcagcgccgagcgctgcgcctcggtcatcttccccgcctggtactggcgccggcggcccaagcgcctgtcggccgtggtgtgcgccctgctgtgggtcctgtccctcctggtcacctgcctgcacaactacttctgcgtgttcctgggccgcggggcccccggcgcggcctgcaggcacatggacatcttcctgggcatcctcctgttcctgctctgctgcccgctcatggtgctgccctgcctggccctcatcctgcacgtggagtgccgggcccgacggcgccagcgctctgccaagctcaaccacgtcatcctggccatggtctccgtcttcctggtgtcctccatctacttagggatcgactggttcctcttctgggtcttccagatcccggcccccttccccgagtacgtcactgacctgtgcatctgcatcaacagcagcgccaagcccatcgtctacttcctggccgggagggacaagtcgcagcggctgtgggagccgctcagggtggtcttccagcgggccctgcgggacggcgctgagctgggggaggccgggggcagcacgcccaacacagtcaccatggagatgcagtgtcccccggggaacgcctcctgagactccagcgcctggaggaggcagtggcaggaatcgtgctcc

SEQ ID NO: 2 - protein, 327 aa, Homo sapiens
MCPGLSEAPELYSRGFLTIEQIAMLPPPAVMNYIFLLLCLCGLVGNGLVLWFFGFSIKRNPFSIYFLHLASADVGYLFSKAVFSILNTGGFLGTFADYIRSVCRVLGLCMFLTGVSLLPAVSAERCASVIFPAWYWRRRPKRLSAVVCALLWVLSLLVTCLHNYFCVFLGRGAPGAACRHMDIFLGILLFLLCCPLMVLPCLALILHVECRARRRQRSAKLNHVILAMVSVFLVSSIYLGIDWFLFWVFQIPAPFPEYVTDLCICINSSAKPIVYFLAGRDKSQRLWEPLRVVFQRALRDGAELGEAGGSTPNTVTMEMQCPPGNAS

SEQ ID NO: 3 - DNA, 25 bp, Artificial Sequence
  Description of Artificial Sequence Primer
gctccacagccactgccttacagac

SEQ ID NO: 4 - DNA, 23 bp, Artificial Sequence
  Description of Artificial Sequence Primer
cacagtcccagggagaagaaggc

SEQ ID NO: 5 - DNA, 29 bp, Artificial Sequence
  Description of Artificial Sequence Primer
acggattcatcacaggtgtccctctctgc

SEQ ID NO: 6 - DNA, 30 bp, Artificial Sequence
  Description of Artificial Sequence Primer
agaagcttggagcacgattcctgccactgc

SEQ ID NO: 7 - DNA, 29 bp, Artificial Sequence
  Description of Artificial Sequence Primer
ggggtaccatcacaggtgtccctctctgc

SEQ ID NO: 8 - DNA, 28 bp, Artificial Sequence
  Description of Artificial Sequence Primer
gctctagagcacgattcctgccactgcc

SEQ ID NO: 9 - DNA, 20 bp, Artificial Sequence
  Description of Artificial Sequence Primer
ccagcagagaatggaaagtc

SEQ ID NO: 10 - DNA, 20 bp, Artificial Sequence
  Description of Artificial Sequence Primer
gatgctgcttacatgtctcg

SEQ ID NO: 11 - DNA, 17 bp, Artificial Sequence
  Description of Artificial Sequence Primer
ggtgaagcaggcgtcgg

SEQ ID NO: 12 - DNA, 21 bp, Artificial Sequence
  Description of Artificial Sequence Primer
gacaaagtggtcgttgagggc

SEQ ID NO: 13 - DNA, 17 bp, Artificial Sequence
  Description of Artificial Sequence Primer
cctggccatggtctccg

SEQ ID NO: 14 - DNA, 16 bp, Artificial Sequence
  Description of Artificial Sequence Primer
acgatgggcttggcgc

SEQ ID NO: 15 - DNA, 29 bp, Artificial Sequence
  Description of Artificial Sequence TaqMan Probe
tggtctcctctgacttcaacagcgacacc

SEQ ID NO: 16 - DNA, 19 bp, Artificial Sequence
  Description of Artificial Sequence TaqMan probe
ccggcccccttccccgagt

(19) United States
(12) Patent Application Publication
(10) Pub. No.: US 2004/0072327 A1
(43) Pub. Date: Apr. 15, 2004

(54) Human g-protein coupled receptor and uses thereof

(76) Inventors: Willy Deleersnijder, CP Weesp (NL); Herman Blockx, CP Weesp (NL); Lucie De Moor, CP Weesp (NL)

Correspondence Address: FINNEGAN, HENDERSON, FARABOW, GARRETT & DUNNER, LLP, 1300 I STREET, NW, WASHINGTON, DC, 20005, US

(21) Appl. No.: 10/398,026

(22) PCT Filed: Sep. 28, 2001
(86) PCT No.: PCT/EP01/11319

(30) Foreign Application Priority Data
Oct. 2, 2000 (EP) 00203411.4

(51) Int. Cl. C12N 1/20

(52) U.S. Cl. 435/252.3

(57) ABSTRACT

The present invention relates to the IGS43 G-protein coupled receptor family, and to polynucleotides encoding said IGS43 proteins. The invention also relates to inhibiting or activating the action of such polynucleotides and polypeptides, to a vector containing said polynucleotides, a host cell containing such vector and non-human transgenic animals where the IGS43-gene is either overexpressed, misexpressed, underexpressed or suppressed (knock-out animals). The invention further relates to a method for screening compounds capable to act as an agonist or an antagonist of said G-protein coupled receptor family IGS43 and the use of IGS43 polypeptides and polynucleotides and agonists or antagonists to the IGS43 receptor family in the treatment of a broad range of disorders and diagnostic assays for such conditions. The invention in particular relates to a method for screening compounds capable to act as an agonist or an antagonist of said G-protein coupled receptor family IGS43 and the use of IGS43 polypeptides and polynucleotides and agonists or antagonists to the IGS43 receptor family in the treatment of dysfunctions, disorders, or diseases related to uterus, kidney, lung, trachea, colon, small intestine, stomach, mammary gland, prostate, testis, central nervous system, cerebellum, and spinal cord, and diagnostic assays for such conditions.

### DESCRIPTION

The present invention relates to novel identified polynucleotides, polypeptides encoded by them and to the use of such polynucleotides and polypeptides, and to their production. More particularly, the polynucleotides and polypeptides of the present invention relate to a G-protein coupled receptor (GPCR), hereinafter referred to as IGS43. The invention also relates to inhibiting or activating the action of such polynucleotides and polypeptides, to a vector containing said polynucleotides, a host cell containing such vector and transgenic animals where the IGS43-gene is either overexpressed, misexpressed, underexpressed and/or suppressed (knockout animals). The invention further relates to a method for screening compounds capable to act as an agonist or an antagonist of said G-protein coupled receptor IGS43.

### BACKGROUND OF THE INVENTION

It is well established that many medically significant biological processes are mediated by proteins participating in signal transduction pathways that involve G-proteins and/or second messengers; e.g., cAMP (Lefkowitz, Nature, 1991, 351:353-354). Herein these proteins are referred to as proteins participating in pathways with G-proteins. Some examples of these proteins include the GPC receptors, such as those for adrenergic agents and dopamine (Kobilka, B. K., et al., Proc. Natl. Acad. Sci., USA, 1987, 84:46-50; Kobilka, B. K., et al., Science, 1987, 238:650-656; Bunzow, J. R., et al., Nature, 1988, 336:783-787), G-proteins themselves, effector proteins, e.g., phospholipase C, adenylate cyclase, and phosphodiesterase, and actuator proteins, e.g., protein kinase A and protein kinase C (Simon, M. I., et al., Science, 1991, 252:802-8).

For example, in one form of signal transduction, upon hormone binding to a GPCR the receptor interacts with the heterotrimeric G-protein and induces the dissociation of GDP from the guanine nucleotide-binding site. At normal cellular concentrations of guanine nucleotides, GTP fills the site immediately. Binding of GTP to the α subunit of the G-protein causes the dissociation of the G-protein from the receptor and the dissociation of the G-protein into a and βγ subunits. The GTP-carrying form then binds to activated adenylate cyclase. Hydrolysis of GTP to GDP, catalyzed by the G-protein itself (α subunit possesses an intrinsic GTPase activity), returns the G-protein to its basal, inactive form. The GTPase activity of the α subunit is, in essence, an internal clock that controls an on/off switch. The GDP bound form of the α subunit has high affinity for βγ and subsequent reassociation of αGDP with βγ returns the system to the basal state. Thus the G-protein serves a dual role, as an intermediate that relays the signal from receptor to effector (in this example adenylate cyclase), and as a clock that controls the duration of the signal.

The membrane bound superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

The G-protein coupled receptor family includes dopamine receptors which bind to neuroleptic drugs used for treating CNS disorders. Other examples of members of this family include, but are not limited to calcitonin, adrenergic, neuropeptideY, somastotatin, neurotensin, neurokinin, capsaicin, VIP, CGRP, CRF, CCK, bradykinin, galanin, motilin, nociceptin, endothelin, CAMP, adenosine, muscarinic, acetylcholine, serotonin, histamine, thrombin, kinin, follicle stimulating hormone, opsin, endothelial differentiation gene-1, rhodopsin, odorant, and cytomegalovirus receptors.

Most G-protein coupled receptors have single conserved cysteine residues in each of the first two extracellular loops which form disulfide bonds that are believed to stabilize functional protein structures. The 7 transmembrane regions are designated as TM1, TM2, TM3, TM4, TM5, TM6 and TM7. The cytoplasmic loop which connects TM5 and TM6 may be a major component of the G-protein binding domain.

Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization.

Recently, it was discovered that certain GPCRs, like the calcitonin-receptor like receptor, might interact with small single pass membrane proteins called receptor activity modifying proteins (RAMP's). This interaction of the GPCR with a certain RAMP is determining which natural ligands have relevant affinity for the GPCR-RAMP combination and regulate the functional signaling activity of the complex (McLathie, L. M. et al., Nature (1998) 393:333-339).

For some receptors, the ligand binding sites of G-protein coupled receptors are believed to comprise hydrophilic sockets formed by several G-protein coupled receptor transmembrane domains, said sockets being surrounded by hydrophobic residues of the G-protein coupled receptors. The hydrophilic side of each G-protein coupled receptor transmembrane helix is postulated to face inward and form a polar ligand-binding site. TM3 has been implicated in several G-protein coupled receptors as having a ligand-binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10:317-331). Different G-protein α-subunits preferentially stimulate particular effectors to modulate various biological functions in a cell. Phosphorylation of cytoplasmic residues of G-protein coupled receptors has been identified as an important mechanism for the regulation of G-protein coupling of some G-protein coupled receptors. G-protein coupled receptors are found in numerous sites within a mammalian host.

Receptors—primarily the GPCR class—have led to more than half of the currently known drugs (Drews, Nature Biotechnology, 1996, 14: 1516). This indicates that these receptors have an established, proven history as therapeutic targets. The new IGS43 GPCR described in this invention dearly satisfies a need in the art for identification and characterization of further receptors that can play a role in diagnosing, preventing, ameliorating or correcting dysfunctions, disorders, or diseases, hereafter generally referred to as “the Diseases”. The Diseases include, but are not limited to, psychiatric and CNS disorders, including schizophrenia, episodic paroxysmal anxiety (EPA) disorders such as obsessive compulsive disorder (OCD), post traumatic stress disorder (PTSD), phobia and panic, major depressive disorder, bipolar disorder, Parkinson's disease, general anxiety disorder, autism, delirium, multiple sclerosis, Alzheimer disease/dementia and other neurodegenerative diseases, severe mental retardation, dyskinesias, Huntington's disease, Tourett's syndrome, tics, tremor, dystonia, spasms, anorexia, bulimia, stroke, addiction/dependency/craving, sleep disorder, epilepsy, migraine; attention deficit/hyperactivity disorder (ADHD); cardiovascular diseases, including heart failure, angina pectoris, arrhythmias, myocardial infarction, cardiac hypertrophy, hypotension, hypertension—e.g. essential hypertension, renal hypertension, or pulmonary hypertension, thrombosis, arteriosclerosis, cerebral vasospasm, subarachnoid hemorrhage, cerebral ischemia, cerebral infarction, peripheral vascular disease, Raynaud's disease, kidney disease—e.g. renal failure; dyslipidemias; obesity; emesis; gastrointestinal disorders, including irritable bowel syndrome (IBS), inflammatory bowel disease (IBD), gastroesophagal reflux disease (GERD), motility disorders and conditions of delayed gastric emptying, such as post operative or diabetic gastroparesis, and diabetes, ulcers—e.g. gastric ulcer; diarrhoea; other diseases including osteoporosis; inflammations; infections such as bacterial, fungal, protozoan and viral infections, particularly infections caused by HIV-1 or HIV-2; pain; cancers; chemotherapy induced injury; tumor invasion; immune disorders; urinary retention; asthma; allergies; arthritis; benign prostatic hypertrophy; endotoxin shock; sepsis; complication of diabetes mellitus; and gynaecological disorders.

In particular, the new IGS43 GPCR described in this invention satisfies a need in the art for identification and characterization of further receptors that can play an important role in diagnosing, preventing, ameliorating or correcting dysfunctions, disorders, or diseases related to uterus, kidney, lung, trachea, colon, small intestine, stomach, mammary gland, prostate, testis, central nervous system, cerebellum, and spinal cord.

### SUMMARY OF THE INVENTION

In one aspect, the invention relates to IGS43 polypeptides, polynucleotides and recombinant materials and methods for their production. Another aspect of the invention relates to methods for using such IGS43 polypeptides, polynucleotides and recombinant materials. Such uses include, but are not limited to, use as a therapeutic target and for treatment of one of the Diseases as mentioned above. In particular the uses include treatment of dysfunctions, disorders, or diseases related to uterus, kidney, lung, trachea, colon, small intestine, stomach, mammary gland, prostate, testis, central nervous system, cerebellum, and spinal cord.

In still another aspect, the invention relates to methods to identify agonists and antagonists using the materials provided by the invention, and treating conditions associated with IGS43 imbalance with the identified compounds. Yet another aspect of the invention relates to diagnostic assays for detecting diseases associated with inappropriate IGS43 activity or levels. A further aspect of the invention relates to animal-based systems which act as models for disorders arising from aberrant expression or activity of IGS43.

### BRIEF DESCRIPTION OF THE FIGURES

FIG. 1. Q-PCR analysis of GAPDH mRNA expression in different human tissues. Reported values (#copies/ng mRNA) represent the average of 2 Q-PCR assays (each assay on independently prepared cDNA). It was assumed that mRNA represents 2% of total RNA and that cDNA synthesis was 100% efficient. As the true efficiency is probably closer to 20-50%, actual copy numbers are likely underestimated 2-5 fold. For tissues marked with “*”, poly(A)⁺ RNA was used whereas for all other tissues total RNA was used.

FIG. 2. Q-PCR analysis of IGS43 mRNA expression in different human tissues. Reported values (#copies/ng mRNA) are from a single determination. It was assumed that mRNA represents 2% of total RNA and that cDNA synthesis was 100% efficient. As the true efficiency is probably closer to 20-50%, actual copy numbers are likely underestimated 2-5 fold. For tissues marked with “*”, poly(A)⁺ RNA was used whereas for all other tissues total RNA was used.

TABLE 1
IGS43-DNA of SEQ ID NO: 1

5′
ATCACAGGTGTCCCTCTCTGCACCTGCGCAGATGTGCCCTGGCCTGAGCG
AGGCCCCGGAACTCTACAGCCGGGGCTTCCTGACCATCGAGCAGATCGCG
ATGCTGCCGCCTCCGGCCGTCATGAACTACATCTTCCTGCTCCTCTGCCT
GTGTGGCCTGGTGGGCAACGGGCTGGTCCTCTGGTTTTTCGGCTTCTCCA
TCAAGAGGAACCCCTTCTCCATCTACTTCCTGCACCTGGCCAGCGCCGAT
GTGGGCTACCTCTTCAGCAAGGCGGTGTTCTCCATCCTGAACACGGGGGG
CTTCCTGGGCACGTTTGCCGACTACATCCGCAGCGTGTGCCGGGTCCTGG
GGCTCTGTATGTTCCTTACCGGCGTGAGCCTCCTGCCGGCCGTCAGCGCC
GAGCGCTGCGCCTCGGTCATCTTCCCCGCCTGGTACTGGCGCCGGCGGCC
CAAGCGCCTGTCGGCCGTGGTGTGCGCCCTGCTGTGGGTCCTGTCCCTCC
TGGTCACCTGCCTGCACAACTACTTCTGCGTGTTCCTGGGCCGCGGGGCC
CCCGGCGCGGCCTGCAGGCACATGGACATCTTCCTGGGCATCCTCCTGTT
CCTGCTCTGCTGCCCGCTCATGGTGCTGCCCTGCCTGGCCCTCATCCTGC
ACGTGGAGTGCCGGGCCCGACGGCGCCAGCGCTCTGCCAAGCTCAACCAC
GTCATCCTGGCCATGGTCTCCGTCTTCCTGGTGTCCTCCATCTACTTAGG
GATCGACTGGTTCCTCTTCTGGGTCTTCCAGATCCCGGCCCCCTTCCCCG
AGTACGTCACTGACCTGTGCATCTGCATCAACAGCAGCGCCAAGCCCATC
GTCTACTTCCTGGCCGGGAGGGACAAGTCGCAGCGGCTGTGGGAGCCGCT
CAGGGTGGTCTTCCAGCGGGCCCTGCGGGACGGCGCTGAGCTGGGGGAGG
CCGGGGGCAGCACGCCCAACACAGTCACCATGGAGATGCAGTGTCCCCCG
GGGAACGCCTCCTGAGACTCCAGCGCCTGGAGGAGGCAGTGGCAGGAATC
GTGCTCC
-3′

TABLE 2
IGS43-protein of SEQ ID NO: 2

MCPGLSEAPELYSRGFLTIEQIAMLPPPAVMNYIFLLLCLCGLVGNGLVL
WFFGFSIKRNPFSIYFLHLASADVGYLFSKAVFSILNTGGFLGTFADYIR
SVCRVLGLCMFLTGVSLLPAVSAERCASVIFPAWYWRRRPKRLSAVVCAL
LWVLSLLVTCLHNYFCVFLGRGAPGAACRHMDIFLGILLFLLCCPLMVLP
CLALILHVECRARRRQRSAKLNHVILAMVSVFLVSSIYLGIDWFLFWVFQ
IPAPFPEYVTDLCICINSSAKPIVYFLAGRDKSQRLWEPLRVVFQRALRD
GAELGEAGGSTPNTVTMEMQCPPGNAS

### DESCRIPTION OF THE INVENTION

Structural and chemical similarity, in the context of sequences and motifs, exists among the IGS43 GPCR of the invention and other human GPCR's. In addition, IGS43 is expressed in uterus, kidney, lung, trachea, colon, small intestine, stomach, mammary gland, prostate, testis, central nervous system, cerebellum, and spinal cord. Therefore, IGS43 is implied to play a role among other things in the Diseases mentioned above. IGS43 in particular is implied to play a role in dysfunctions, disorders, or diseases related to uterus, kidney, lung, trachea, colon, small intestine, stomach, mammary gland, prostate, testis, central nervous system, cerebellum, and spinal cord.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited in this specification are herein incorporated by reference as if each individual publication were specifically and individually indicated to be incorporated by reference herein as though fully set forth.

Definitions

The following definitions are provided to facilitate understanding of certain terms used frequently herein.

“IGS43” refers, among others, to a polypeptide consisting of the amino acid sequence set forth in SEQ ID NO:2, or a Variant thereof.

“Receptor Activity” or “Biological Activity of the Receptor” refers to the metabolic or physiologic function of said IGS43 including similar activities or improved activities or these activities with decreased undesirable side effects. Also included are antigenic and immunogenic activities of said IGS43.

“IGS43-gene” refers to a polynucleotide consisting of the nucleotide sequence set forth in SEQ ID NO:1 or respective Variants, e.g. allelic Variants, thereof and/or their complements.

“Antibodies” as used herein includes polyclonal and monoclonal antibodies, chimeric, single chain, and humanized antibodies, as well as Fab fragments, including the products of a Fab or other immunoglobulin expression library.

“Isolated” means altered “by the hand of man” from the natural state and/or separated from the natural environment. Thus, if an “isolated” composition or substance that occurs in nature has been “isolated”, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single-and double-stranded regions, hybrid molecules comprising DNA and RNA that may be single-stranded or, more typically, double-stranded or a mixture of single- and double-stranded regions. In addition, “polynucleotide” may also include triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to short chains, commonly referred to as peptides, oligopeptides or oligomers, and to longer chains, generally referred to as proteins, and/or to combinations thereof. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well-described in basic texts and in more detailed monographs, as well as in voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol; cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth. Enzymol. (1990) 182:626-646 and Rattan et al., “Protein Synthesis: Posttranslational Modifications and Aging”, Ann. NY Acad. Sci. (1992) 663:48-62.

“Variant” as the term is used herein, is a polynucleotide or polypeptide that differs from a reference polynucleotide or polypeptide respectively, but retains essential properties such as essential biological, structural, regulatory or biochemical properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” is a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed.; Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J. Applied Math. (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J. Applied Math. (1988) 48:1073. Methods to determine identity and similarity are codified in computer programs. Preferred computer program methods to determine identity and similarity between two sequences include, but are not limited to, GCG program package (Devereux, J., et al., Nucleic Acids Research (1984) 12(1):387), BLASTP, BLASTN, FASTA (Atschul, S. F. et al., J. Molec. Biol. (1990) 215:403). The word “homology” may substitute for the word “identity”.

As an illustration, by a polynucleotide having a nucleotide sequence having at least, for example, 95% “identity” to a reference nucleotide sequence of SEQ ID NO: 1 is intended that the nucleotide sequence of the polynucleotide is identical to the reference sequence except that the polynucleotide sequence may include up to five nucleotide differences per each 100 nucleotides of the reference nucleotide sequence of SEQ ID NO: 1. In other words, to obtain a polynucleotide having a nucleotide sequence at least 95% identical to a reference nucleotide sequence, up to any 5% of the nucleotides in the reference sequence may be deleted or substituted with another nucleotide, or a number of nucleotides up to any 5% of the total nucleotides in the reference sequence may be inserted into the reference sequence, or in a number of nucleotides of up to any 5% of the total nucleotides in the reference sequence there may be a combination of deletion, insertion and substitution. These differences may occur at the 5′ or 3′ terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO:2 is intended that the amino acid sequence of the polypeptide is identical to the reference sequence except that the polypeptide sequence may include up to five amino acid alterations per each 100 amino acids of the reference amino acid of SEQ ID NO: 2. In other words, to obtain a polypeptide having an amino acid sequence at least 95% identical to a reference amino acid sequence, up to any 5% of the amino acid residues in the reference sequence may be deleted or substituted with another amino acid, or a number of amino acids up to any 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

Polypeptides of the Invention

In one aspect, the present invention relates to IGS43 polypeptides (including IGS43 proteins). The IGS43 polypeptides include the polypeptide of SEQ ID NO:2 and the polypeptide having the amino acid sequence encoded by the DNA insert contained in the deposit no. CBS 109714; deposited on Aug. 30, 2001 at the Centraalbureau voor Schimmelcultures at Utrecht (the Netherlands), as well as polypeptides comprising the amino acid sequence of SEQ ID NO:2 and the polypeptide having the amino acid sequence encoded by the DNA insert contained in the deposit no. CBS 109714 at the Centraalbureau voor Schimmelcultures at Utrecht (the Netherlands), and polypeptides comprising an amino acid sequence having at least 80% identity to that of SEQ ID NO:2 and/or to the polypeptide having the amino acid sequence encoded by the DNA insert contained in the deposit no. CBS 109714 at the Centraalbureau voor Schimmelcultures at Utrecht (the Netherlands) over its entire length, and still more preferably at least 90% identity, and even still more preferably at least 95% identity to said amino acid sequence. Furthermore, those with at least 97%, in particular at least 99%, are highly preferred. Also included within IGS43 polypeptides are polypeptides having the amino acid sequence which has at least 80% identity to the polypeptide having the amino acid sequence of SEQ ID NO: 2 or the polypeptide having the amino acid sequence encoded by the DNA insert contained in the deposit no. CBS 109714 at the Centraalbureau voor Schimmelcultures at Utrecht (the Netherlands) over its entire length, and still more preferably at least 90% identity, and even still more preferably at least 95% identity to SEQ ID NO: 2. Furthermore, those with at least 97%, in particular at least 99% are highly preferred. Preferably IGS43 polypeptides exhibit at least one biological activity of the receptor.

Particularly preferred is an isolated IGS43 polypeptide consisting of an amino acid sequence which is at least 95.5% identical to the amino acid sequence of SEQ ID NO:2 or to the polypeptide encoded by the DNA insert contained in the deposit no. CBS 109714 at the Centraalbureau voor Schimmelcultures at Utrecht (The Netherlands) over its entire length.

In an additional embodiment of the invention, the IGS43 polypeptides may be a part of a larger protein such as a fusion protein. It is often advantageous to include an additional amino acid sequence which contains secretory or leader sequences, pro-sequences, sequences which aid in purification such as multiple histidine residues, sequences which aid in detection such as antigenic peptide tags (such as the haemagglutinin (HA) tag), or an additional sequence for stability during recombinant production.

Fragments of the IGS43 polypeptides are also included in the invention. A fragment is a polypeptide having an amino acid sequence that is the same as part of, but not all of, the amino acid sequence of the aforementioned IGS43 polypeptides. As with IGS43 polypeptides, fragments may be “free-standing,” or comprised within a larger polypeptide of which they form a part or region, most preferably as a single continuous region. Representative examples of polypeptide fragments of the invention, include, for example, fragments from about amino acid number 1-20; 21-40, 41-60, 61-80, 81-100; and 101 to the end of IGS43 polypeptide. In this context “about” includes the particularly recited ranges larger or smaller by several, 5, 4, 3, 2 or 1 amino acid at either extreme or at both extremes.

Preferred fragments include, for example, truncation polypeptides having the amino acid sequence of IGS43 polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate receptor activity, including those with a similar activity or an improved activity, or with a decreased undesirable activity. Also included are those that are antigenic or immunogenic in an animal, especially in a human.

Thus, the polypeptides of the invention include polypeptides having an amino acid sequence that is at least 80% identical to either that of SEQ ID NO:2 and/or the polypeptide having the amino acid sequence encoded by the DNA insert contained in the deposit no. CBS 109714 at the Centraalbureau voor Schimmelcultures at Utrecht (the Netherlands), or fragments thereof with at least 80% identity to the corresponding fragment. Preferably, all of these polypeptide fragments retain the biological activity of the receptor, including antigenic activity. Variants of the defined sequence and fragments also form part of the present invention. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gin; and among the basic residues Lys and Arg; or aromatic residues Phe and Tyr. Particularly preferred are variants in which several, 5-10, 1-5, or 1-2 amino acids are substituted, deleted, or added in any combination.

The IGS43 polypeptides of the invention can be prepared in any suitable manner. Such polypeptides include isolated naturally occurring polypeptides, recombinantly produced polypeptides, synthetically produced polypeptides, or polypeptides produced by a combination of these methods. Methods for preparing such polypeptides are well known in the art

Polynucleotides of the Invention

A further aspect of the invention relates to IGS43 polynucleotides. IGS43 polynucleotides include isolated polynucleotides which encode the IGS43 polypeptides and fragments, and polynucleotides closely related thereto. More specifically, the IGS43 polynucleotide of the invention includes a polynucleotide comprising the nucleotide sequence contained in SEQ ID NO:1, such as the one capable of encoding a IGS43 polypeptide of SEQ ID NO: 2, polynucleotides having the particular sequence of SEQ ID NO: 1 and polynucleotides which essentially correspond to the DNA insert contained in the deposit no. CBS 109714 at the Centraalbureau voor Schimmelcultures at Utrecht (the Netherlands).

IGS43 polynucleotides further include polynucleotides comprising a nucleotide sequence that has at least 80% identity over its entire length to a nucleotide sequence encoding the IGS43 polypeptide of SEQ ID NO:2, polynucleotides comprising a nucleotide sequence that is at least 80% identical to that of SEQ ID NO:1 over its entire length and a polynucleotide which essentially corresponds to the DNA insert contained in the deposit no. CBS 109714 at the Centraalbureau voor Schimmelcultures at Utrecht (the Netherlands).

In this regard, polynucleotides with at least 90% identity are particularly preferred, and those with at least 95% are especially preferred. Furthermore, those with at least 97% are highly preferred and those with at least 98-99% are most highly preferred, with at least 99% being the most preferred. Also included under IGS43 polynucleotides are a nucleotide sequence which has sufficient identity to a nucleotide sequence contained in SEQ ID NO: 1 or to the DNA insert contained in the deposit no. CBS 109714 at the Centraalbureau voor Schimmelcultures at Utrecht (the Netherlands) to hybridize under conditions useable for amplification or for use as a probe or marker. The invention also provides polynucleotides which are complementary to such IGS43 polynucleotides.

Particularly preferred is an isolated IGS43 polynucleotide selected from the group consisting of.

1. a nucleotide sequence encoding the IGS43 polypeptide according to SEQ ID NO: 2;

2. a nucleotide sequence encoding the polypeptide encoded by the DNA insert contained in the deposit no. CBS 109714 at the Centraalbureau voor Schimmelcultures at Utrecht (The Netherlands), in particular a nucleotide sequence corresponding to the SEQ ID NO: 1;

3. a nucleotide sequence having at least 95.5% (preferably at least 96%) sequence identity over its entire length to the nucleotide sequence of (a) or (b);

4. a nucleotide sequence which is complimentary to the nucleotide sequence of (a) or (b) or (c).

IGS43 of the invention is structurally related to other proteins of the G-protein coupled receptor family, as shown by the results of BLAST searches in the public databases. The amino acid sequence of Table 2 (SEQ ID NO:2) was most similar with the rat GPCR RTA (85% identities over 327 aligned residues; Swissprot accession no P23749). In the patent literature the application EP1067182 Seq Id 322 gives a protein for a probable G-protein coupled receptor. This protein is 16 amino acids longer, but otherwise identical to the IGS43 protein. Thus, IGS43 polypeptides and polynucleotides of the present invention are expected to have, inter alia, similar biological functions/properties to their homologous polypeptides and polynucleotides, and their utility is obvious to anyone skilled in the art.

Polynucleotides of the invention can be obtained from natural sources such as genomic DNA In particular, degenerated PCR primers can be designed that encode conserved regions within a particular GPCR gene subfamily. PCR amplification reactions on genomic DNA or cDNA using the degenerate primers will result in the amplification of several members (both known and novel) of the gene family under consideration (the degenerated primers must be located within the same exon, when a genomic template is used). (Libert et al., Science, 1989, 244: 569-572). Polynucleotides of the invention can also be synthesized using well-known and commercially available techniques (e.g. F. M. Ausubel et al, 2000, Current Protocols in Molecular Biology).

The nucleotide sequence encoding the IGS43 polypeptide of SEQ ID NO:2 may be identical to the polypeptide encoding sequence contained in SEQ ID NO:1 (nucleotide number 32 to 1012), or it may be a different nucleotide sequence, which as a result of the redundancy (degeneracy) of the genetic code might also show alterations compared to the polypeptide encoding sequence contained in SEQ ID NO:1, but also encodes the polypeptide of SEQ ID NO:2.

When the polynucleotides of the invention are used for the recombinant production of the IGS43 polypeptide, the polynucleotide may include the coding sequence for the mature polypeptide or a fragment thereof, by itself; the coding sequence for the mature polypeptide or fragment in reading frame with other coding sequences, such as those encoding a leader or secretory sequence, a pre-, or pro- or prepro-protein sequence, or other fusion peptide portions. For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc. Natl. Acad. Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Further preferred embodiments are polynucleotides encoding IGS43 variants comprising the amino acid sequence of the IGS43 polypeptide of SEQ ID NO:2 in which several, 5-10, 1-5, 1-3,1-2 or 1 amino add residues are substituted, deleted or added, in any combination.

The polynucleotides of the invention can be engineered using methods generally known in the art in order to alter IGS43-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create amino acid substitutions, create new restriction sites, alter modification (e.g. glycosylation or phosphorylation) patterns, change codon preference, produce splice variants, and so forth.

The present invention further relates to polynucleotides that hybridize to the herein above-described sequences. In this regard, the present invention especially relates to polynucleotides which hybridize under stringent conditions to the polynucleotides described above. As herein used, the term “stringent conditions” means hybridization will occur only if there is at least 80%, and preferably at least 90%, and more preferably at least 95%, yet even more preferably at least 97%, in particular at least 99% identity between the sequences.

Polynucleotides of the invention, which are identical or sufficiently identical to a nucleotide sequence contained in SEQ ID NO:1 or a fragment thereof, may be used as hybridization probes for cDNA and genomic DNA, to isolate full-length cDNAs and genomic clones encoding IGS43 and to isolate cDNA and genomic clones of other genes (including genes encoding homologs and orthologs from species other than human) that have a high sequence similarity to the IGS43 gene. People skilled in the art are well aware of such hybridization techniques. Typically these nucleotide sequences are 80% identical, preferably 90% identical, more preferably 95% identical to that of the referent The probes generally will comprise at least 5 nucleotides, and preferably at least 8 nucleotides, and more preferably at least 10 nucleotides, yet even more preferably at least 12 nucleotides, in particular at least 15 nucleotides. Most preferred, such probes will have at least 30 nucleotides and may have at least 50 nucleotides. Particularly preferred probes will range between 30 and 50 nucleotides.

One embodiment, to obtain a polynucleotide encoding the IGS43 polypeptide, including homologs and orthologs from species other than human, comprises the steps of screening an appropriate library under stringent hybridization conditions with a labeled probe having the SEQ ID NO: 1 or a fragment thereof, and isolating full-length cDNA and genomic clones containing said polynucleotide sequence. Such hybridization techniques are well known to those of skill in the art. Stringent hybridization conditions are as defined above or alternatively conditions under overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate (w/v), and 20 microgram/ml denatured, sheared salmon sperm DNA, followed by washing the filters in 0.1×SSC at about 65° C.

The polynucleotides and polypeptides of the present invention may be used as research reagents and materials for discovery of treatments and diagnostics to animal and human disease.

Vectors, Host Cells, Expression

The present invention also relates to vectors which comprise a polynucleotide or polynucleotides of the present invention, and host cells which are genetically engineered with vectors of the invention and to the production of polypeptides of the invention by recombinant techniques. Cell-free translation systems can also be used to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., BASIC METHODS IN MOLECULAR BIOLOGY (1986) and Sambrook et al., MOLECULAR CLONING: A LABORATORY MANUAL, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

For secretion of the translated protein into the lumen of the endoplasmic reticulum, into the periplasmic space or into the extracellular environment, appropriate secretion signals may be incorporated into the desired polypeptide. These signals may be endogenous to the polypeptide or they may be heterologous signals, i.e. derived from a different species.

If the IGS43 polypeptide is to be expressed for use in screening assays, generally, it is preferred that the polypeptide be produced at the surface of the cell. In this event, the cells may be harvested prior to use in the screening assay. In case the affinity or functional activity of the IGS43 polypeptide is modified by receptor activity modifying proteins (RAMP), coexpression of the relevant RAMP most likely at the surface of the cell is preferred and often required. Also in this event harvesting of cells expressing the IGS43 polypeptide and the relevant RAMP prior to use in screening assays is required. If the IGS43 polypeptide is secreted into the medium, the medium can be recovered in order to recover and purify the polypeptide; if produced intracellularly, the cells must first be lysed before the polypeptide is recovered. Membranes expressing the IGS43 polypeptide can be recovered by methods that are well known to a person skilled in the art. In general, such methods include harvesting of the cells expressing the IGS43 polypeptide and homogenization of the cells by a method such as, but not limited to, pottering. The membranes may be recovered by washing the suspension one or several times.

IGS43 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well-known techniques for refolding proteins may be employed to regenerate active conformation when the polypeptide is denatured during isolation and or purification.

Diagnostic Assays

This invention also relates to the use of IGS43 polynucleotides for use as diagnostic reagents. Detection of a mutated form of the IGS43 gene associated with a dysfunction will provide a diagnostic tool that can add to or define a diagnosis of a disease or susceptibility to a disease which results from under-expression, over-expression or altered expression of IGS43. Also in this event co-expression of relevant receptor activity modifying proteins can be required to obtain diagnostic assays of desired quality. Individuals carrying mutations in the IGS43 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled IGS43 nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science (1985) 230:1242. Sequence changes at specific locations may also be revealed by nuclease protection assays, such as RNase and S1 protection or the chemical cleavage method. See Cotton et al., Proc. Natl. Acad. Sci. USA (1985) 85: 4397-4401. In another embodiment, an array of oligonucleotide probes comprising the IGS43 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al., Science, Vol 274, pp 610-613 (1996)).

The diagnostic assays offer a process for diagnosing or determining a susceptibility to among other things the Diseases as mentioned above, through detection of mutation in the IGS43 gene by the methods described. The diagnostic assays in particular offer a process for diagnosing or determining a susceptibility to dysfunctions, disorders, or diseases related to uterus, kidney, lung, trachea, colon, small intestine, stomach, mammary gland, prostate, testis, central nervous system, cerebellum, and spinal cord, through detection of mutation in the IGS43 gene by the methods described.

In addition, among other things, the Diseases as mentioned above can be diagnosed by methods comprising determining from a sample derived from a subject an abnormally decreased or increased level of the IGS43 polypeptide or IGS43 mRNA In particular dysfunctions, disorders, or diseases related to uterus, kidney, lung, trachea, colon, small intestine, stomach, mammary gland, prostate, testis, central nervous system, cerebellum, and spinal cord, can be diagnosed by methods comprising determining from a sample derived from a subject an abnormally decreased or increased level of the IGS43 polypeptide or IGS43 mRNA.

Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as an IGS43, in a sample derived from a host are well known to those of skill in the art. Such assay methods include radioimmunoassays, competitive-binding assays, Western Blot analysis and ELISA assays.

In another aspect, the present invention relates to a diagnostic kit for among other things the Diseases or suspectability to one of the Diseases as mentioned above. In particular, the present invention relates to a diagnostic kit for dysfunctions, disorders, or diseases related to uterus, kidney, lung, trachea, colon, small intestine, stomach, mammary gland, prostate, testis, central nervous system, cerebellum, and spinal cord.

The kit may comprise:

(a) an IGS43 polynucleotide, preferably the nucleotide sequence of SEQ ID NO:1, or a fragment thereof, and/or

(b) a nucleotide sequence complementary to that of (a); and/or

(c) an IGS43 polypeptide, preferably the polypeptide of SEQ ID NO:2, or a fragment thereof, and/or

(d) an antibody to an IGS43 polypeptide, preferably to the polypeptide of SEQ ID NO: 2; and/or

(e) a RAMP polypeptide required for the relevant biological or antigenic properties of an IGS43 polypeptide.

It will be appreciated that in any such kit, (a), (b), (c) (d) or (e) may comprise a substantial component.

Chromosome Assays

The nucleotide sequences of the present invention are also valuable for chromosome identification. The sequence is specifically targeted to and can hybridize with a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

Antibodies

The polypeptides of the invention or their fragments or analogs thereof, or cells expressing them if required together with relevant RAMP's, may also be used as immunogens to produce antibodies immunospecific for the IGS43 polypeptides. The term “immunospecific” means that the antibodies have substantial greater affinity for the polypeptides of the invention than their affinity for other related polypeptides in the prior art.

Antibodies generated against the IGS43 polypeptides may be obtained by administering the polypeptides or epitope-bearing fragments, analogs or cells to an animal, preferably a nonhuman, using routine protocols. For preparation of monoclonal antibodies, any technique, which provides antibodies produced by continuous cell line cultures, may be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., MONOCLONAL ANTIBODIES AND CANCER THERAPY, pp. 77-96, Alan R. Liss, Inc., 1985).

The above-described antibodies may be employed to isolate or to identify clones expressing the polypeptide or to purify the polypeptides by affinity chromatography.

Antibodies against IGS43 polypeptides as such, or against IGS43 polypeptide-RAMP complexes, may also be employed to treat among other things the Diseases as mentioned above. In particular, antibodies against IGS43 polypeptides as such, or against IGS43 polypeptide-RAMP complexes, may be employed to treat dysfunctions, disorders, or diseases related to uterus, kidney, lung, trachea, colon, small intestine, stomach, mammary gland, prostate, testis, central nervous system, cerebellum, and spinal cord.

Animals

Another aspect of the invention relates to non-human animal-based systems which act as models for disorders arising from aberrant expression or activity of IGS43. Non-human animal-based model systems may also be used to further characterize the activity of the IGS43 gene. Such systems may be utilized as part of screening strategies designed to identify compounds which are capable to treat IGS43 based disorders such as among other things the Diseases as mentioned above. In particular, the systems may be utilized as part of screening strategies designed to identify compounds which are capable to treat IGS43 based dysfunctions, disorders, or diseases related to uterus, kidney, lung, trachea, colon, small intestine, stomach, mammary gland, prostate, testis, central nervous system, cerebellum, and spinal cord.

In this way the animal-based models may be used to identify pharmaceutical compounds, therapies and interventions which may be effective in treating disorders of aberrant expression or activity of IGS43. In addition such animal models may be used to determine the LD₅₀ and the ED₅₀ in animal subjects. These data may be used to determine the in vivo efficacy of potential IGS43 disorder treatments.

Animal-based model systems of IGS43 based disorders, based on aberrant IGS43 expression or activity, may include both non-recombinant animals as well as recombinantly engineered transgenic animals.

Animal models for IGS43 disorders may include, for example, genetic models. Animal models exhibiting IGS43 based disorder-like symptoms may be engineered by utilizing, for example, IGS43 sequences such as those described, above, in conjunction with techniques for producing transgenic animals that are well known to persons skilled in the art. For example, IGS43 sequences may be introduced into, and overexpressed and/or misexpressed in, the genome of the animal of interest, or, if endogenous IGS43 sequences are present, they may either be overexpressed, misexpressed, or, alternatively, may be disrupted in order to underexpress or inactivate IGS43 gene expression.

In order to overexpress or misexpress a IGS43 gene sequence, the coding portion of the IGS43 gene sequence may be ligated to a regulatory sequence which is capable of driving high level gene expression or expression in a cell type in which the gene is not normally expressed in the animal type of interest Such regulatory regions will be well known to those skilled in the art, and may be utilized in the absence of undue experimentation.

For underexpression of an endogenous IGS43 gene sequence, such a sequence may be isolated and engineered such that when reintroduced into the genome of the animal of interest, the endogenous IGS43 gene alleles will be inactivated, or “knocked-out”. Preferably, the engineered IGS43 gene sequence is introduced via gene targeting such that the endogenous IGS43 sequence is disrupted upon integration of the engineered IGS43 gene sequence into the animal's genome.

Animals of any species, including, but not limited to, mice, rats, rabbits, squirrels, guinea-pigs, pigs, micro-pigs, goats, and non-human primates, e.g., baboons, monkeys, and chimpanzees may be used to generate animal models of IGS43 related disorders.

Any technique known in the art may be used to introduce a IGS43 transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (van der Putten et al., Proc. Natl. Acad. Sci., USA 82:6148-6152, 1985); gene targeting in embryonic stem cells (Thompson et al., Cell 56:313-321, 1989,); electroporation of embryos (Lo, Mol. Cell. Biol. 3:1803-1B14, 1983); and sperm-mediated gene transfer (Lavitrano et al., Cell 57:717-723, 1989); etc. For a review of such techniques, see Gordon, Transgenic Animals, Intl. Rev. Cytol.115:171-229, 1989.

The present invention provides for transgenic animals that carry the IGS43 transgene in all their cells, as well as animals which carry the transgene in some, but not all their cells, i.e., mosaic animals. (See, for example, techniques described by Jakobovits, Curr. Biol. 4:761-763, 1994) The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., Proc. Natl. Acad. Sci. USA 89:6232-6236, 1992).

The regulatory sequences required for such a cell-type specific activation will depend upon the particular cell type of interest, and will be apparent to those of skill in the art.

When it is desired that the IGS43 transgene be integrated into the chromosomal site of the endogenous IGS43 gene, gene targeting is preferred. Briefly, when such a technique is to be utilized, vectors containing some nucleotide sequences homologous to the endogenous IGS43 gene of interest (e.g., nucleotide sequences of the mouse IGS43 gene) are designed for the purpose of integrating, via homologous recombination with chromosomal sequences, into and disrupting the function of, the nucleotide sequence of the endogenous IGS43 gene or gene allele. The transgene may also be selectively introduced into a particular cell type, thus inactivating the endogenous gene of interest in only that cell type, by following, for example, the teaching of Gu et al. (Gu, H. et al.-, Science 265:103-106, 1994). The regulatory sequences required for such a cell-type specific inactivation will depend upon the particular cell type of interest, and will be apparent to those of skill in the art

Once transgenic animals have been generated, the expression of the recombinant IGS43 gene and protein may be assayed utilizing standard techniques. Initial screening may be accomplished by Southern blot analysis or PCR techniques to analyze animal tissues to assay whether integration of the transgene has taken place. The level of mRNA expression of the IGS43 transgene in the tissues of the transgenic animals may also be assessed using techniques which include but are not limited to Northern blot analysis of tissue samples obtained from the animal, in situ hybridization analysis, and RT-PCR. Samples of target gene-expressing tissue, may also be evaluated immunocytochemically using antibodies specific for the target gene transgene product of interest. The IGS43 transgenic animals that express IGS43 gene mRNA or IGS43 transgene peptide (detected immunocytochemically, using antibodies directed against target gene product epitopes) at easily detectable levels may then be further evaluated to identify those animals which display characteristic IGS43 based disorder symptoms.

Once IGS43 transgenic founder animals are produced (i.e., those animals which express IGS43 proteins in cells or tissues of interest, and which, preferably, exhibit symptoms of IGS43 based disorders), they may be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound IGS43 transgenics that express the IGS43 transgene of interest at higher levels because of the effects of additive expression of each IGS43 transgene; crossing of heterozygous transgenic animals to produce animals homozygous for a given integration site in order to both augment expression and eliminate the possible need for screening of animals by DNA analysis; crossing of separate homozygous lines to produce compound heterozygous or homozygous lines; breeding animals to different inbred genetic backgrounds so as to examine effects of modifying alleles on expression of the IGS43 transgene and the development of IGS43-like symptoms. One such approach is to cross the IGS43 transgenic founder animals with a wild type strain to produce an F1 generation that exhibits IGS43 related disorder-like symptoms, such as those described above. The F1 generation may then be inbred in order to develop a homozygous line, if it is found that homozygous target gene transgenic animals are viable.

Vaccines

Another aspect of the invention relates to a method for inducing an immunological response in a mammal which comprises administering to (for example by inoculation) the mammal the IGS43 polypeptide, or a fragment thereof, if required together with a RAMP polypeptide, adequate to produce antibody and/or T cell immune response to protect said animal from among other things one of the Diseases as mentioned above. In particular, the invention relates to a method for inducing an immunological response in a mammal which comprises administering to (for example by inoculation) the mammal the IGS43 polypeptide, or a fragment thereof, if required together with a RAMP polypeptide, adequate to produce antibody and/or T cell immune response to protect said animal from dysfunctions, disorders, or diseases related to uterus, kidney, lung, trachea, colon, small intestine, stomach, mammary gland, prostate, testis, central nervous system, cerebellum, and spinal cord.

Yet another aspect of the invention relates to a method of inducing immunological response in a mammal which comprises delivering the IGS43 polypeptide via a vector directing expression of the IGS43 polynucleotide in vivo in order to induce such an immunological response to produce antibody to protect said animal from diseases.

A further aspect of the invention relates to an immunological/vaccine formulation (composition) which, when introduced into a mammalian host, induces an immunological response in that mammal to an IGS43 polypeptide wherein the composition comprises an IGS43 polypeptide or IGS43 gene. Such immunological/vaccine formulations (compositions) may be either therapeutic immunological/vaccine formulations or prophylactic immunological/vaccine formulations. The vaccine formulation may further comprise a suitable carrier. Since the IGS43 polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Screening Assays

The IGS43 polypeptide of the present invention may be employed in a screening process for compounds which bind the receptor and which activate (agonists) or inhibit activation of (antagonists) the receptor polypeptide of the present invention. Thus, polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics.

IGS43 polypeptides are responsible for biological functions, including pathologies. Accordingly, it is desirable to find compounds and drugs which stimulate IGS43 on the one hand and which can inhibit the function of IGS43 on the other hand. In general, agonists are employed for therapeutic and prophylactic purposes for such conditions as among other things the Diseases as mentioned above. In particular, agonists are employed for therapeutic and prophylactic purposes for dysfunctions, disorders, or diseases related to uterus, kidney, lung, trachea, colon, small intestine, stomach, mammary gland, prostate, testis, central nervous system, cerebellum, and spinal cord.

Antagonists may be employed for a variety of therapeutic and prophylactic purposes for such conditions as among other things the Diseases as mentioned above. In particular, antagonists may be employed for a variety of therapeutic and prophylactic purposes for dysfunctions, disorders, or diseases related to uterus, kidney, lung, trachea, colon, small intestine, stomach, mammary gland, prostate, testis, central nervous system, cerebellum, and spinal cord.

In general, such screening procedures involve producing appropriate cells, which express the receptor polypeptide of the present invention on the surface thereof and, if essential coexpression of RAMP's at the surface thereof. Such cells include cells from mammals, yeast, Drosophila or E. coli. Cells expressing the receptor (or cell membrane containing the expressed receptor) are then contacted with a test compound to observe binding, or stimulation or inhibition of a functional response.

One screening technique includes the use of cells which express the receptor of this invention (for example, transfected CHO cells) in a system which measures extracellular pH, intracellular pH, or intracellular calcium changes caused by receptor activation. In this technique, compounds may be contacted with cells expressing the receptor polypeptide of the present invention. A second messenger response, e.g., signal transduction, pH changes, or changes in calcium level, is then measured to determine whether the potential compound activates or inhibits the receptor.

Another method involves screening for receptor inhibitors by determining modulation of a receptor-mediated signal, such as CAMP accumulation and/or adenylate cyclase activity. Such a method involves transfecting an eukaryotic cell with the receptor of this invention to express the receptor on the cell surface. The cell is then exposed to an agonist to the receptor of this invention in the presence of a potential antagonist. If the potential antagonist binds the receptor, and thus inhibits receptor binding, the agonist-mediated signal will be modulated.

Another method for detecting agonists or antagonists for the receptor of the present invention is the yeast-based technology as described in U.S. Pat. No. 5,482,835.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing an IGS43 polypeptide to form a mixture, measuring the IGS43 activity in the mixture, and comparing the IGS43 activity of the mixture to a standard.

The IGS43 cDNA, protein and antibodies to the protein may also be used to configure assays for detecting the effect of added compounds on the production of IGS43 mRNA and protein in cells. For example, an ELISA may be constructed for measuring secreted or cell associated levels of IGS43 protein using monoclonal and polyclonal antibodies by standard methods known in the art, and this can be used to discover agents which may inhibit or enhance the production of IGS43 (also called antagonist or agonist, respectively) from suitably manipulated cells or tissues. Standard methods for conducting screening assays are well known in the art.

Examples of potential IGS43 antagonists include antibodies or, in some cases, oligonucleotides or proteins which are closely related to the ligand of the IGS43, e.g., a fragment of the ligand, or small molecules which bind to the receptor but do not elicit a response, so that the activity of the receptor is prevented.

Thus in another aspect, the present invention relates to a screening kit for identifying agonists, antagonists, ligands, receptors, substrates, enzymes, etc. for IGS43 polypeptides; or compounds which decrease, increase and/or otherwise enhance the production of IGS43 polypeptides, which comprises:

(a) an IGS43 polypeptide, preferably that of SEQ ID NO:2;

(b) a recombinant cell expressing an IGS43 polypeptide, preferably that of SEQ ID NO:2;

(c) a cell membrane expressing an IGS43 polypeptide, preferably that of SEQ ID NO:2; or

(d) antibody to an IGS43 polypeptide, preferably that of SEQ ID NO: 2.

It will be appreciated that in any such kit, (a), (b), (c) or (d) may comprise a substantial component.

Prophylactic and Therapeutic Methods

This invention provides methods of treating abnormal conditions related to both an excess of and insufficient amounts of IGS43 activity.

If the activity of IGS43 is in excess, several approaches are available. One approach comprises administering to a subject an inhibitor compound (antagonist) as hereinabove described along with a pharmaceutically acceptable carrier in an amount effective to inhibit activation by blocking binding of ligands to the IGS43, or by inhibiting interaction with a RAMP polypeptide or a second signal, and thereby alleviating the abnormal condition.

In another approach, soluble forms of IGS43 polypeptides still capable of binding the ligand in competition with endogenous IGS43 may be administered. Typical embodiments of such competitors comprise fragments of the IGS43 polypeptide.

In still another approach, expression of the gene encoding endogenous IGS43 can be inhibited using expression-blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. USA (1988). Alternatively, oligonucleotides, which form triple helices with the gene, can be supplied. See, for example, Lee et al., Nucleic Acids Res (1979) 6:3073; Cooney et al., Science (1988) 241:456; Dervan et al, Science (1991) 251:1360. These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesized with these or other modified backbones also form part of the present invention.

In addition, expression of the IGS43 polypeptide may be prevented by using ribozymes specific to the IGS43 mRNA sequence. Ribozymes are catalytically active RNAs that can be natural or synthetic (see for example Usman, N, et al., Curr. Opin. Struct. Biol (1996) 6(4), 527-33.) Synthetic ribozymes can be designed to specifically cleave IGS43 mRNAs at selected positions thereby preventing translation of the IGS43 mRNAs into functional polypeptide. Ribozymes may be synthesized with a natural ribose phosphate backbone and natural bases, as normally found in RNA molecules. Alternatively the ribosymes may be synthesized with non-natural backbones to provide protection from ribonuclease degradation, for example, 2′-O-methyl RNA, and may contain modified bases.

For treating abnormal conditions related to an under-expression of IGS43 and its activity, several approaches are also available. One approach comprises administering to a subject a therapeutically effective amount of a compound which activates IGS43, i.e., an agonist as described above, in combination with a pharmaceutically acceptable carrier, to thereby alleviate the abnormal condition. Alternatively, gene therapy may be employed to effect the endogenous production of IGS43 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, Strachan T. and Read A. P., BIOS Scientific Publishers Ltd (1996).

Any of the therapeutic methods described above may be applied to any subject in need of such therapy, including, for example, mammals such as dogs, cats, cows, horses, rabbits, monkeys, and most preferably, humans.

Formulation and Administration

Peptides, such as the soluble form of IGS43 polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible.

The dosage range required depends on the choice of peptide or compound, the route of administration, the nature of the formulation, the nature of the subjects condition, and the judgment of the attending practitioner. Suitable dosages are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

The following examples are only intended to further illustrate the invention in more detail, and therefore these examples are not deemed to restrict the scope of the invention in any way.

### EXAMPLE 1

### The cloning of cDNA Encoding a Novel G Protein-Coupled Receptor

In the public domain databank of unfinished high throughput genomic DNA sequences (htgs) we identified a genomic sequence (accession n^{o} AC019124) which potentially encoded a novel G-protein coupled receptor (GPCR). We refer to this novel GPCR as IGS43. It was decided to investigate whether this genomic sequence represented a functional gene by trying to clone its cDNA from human tissues. Human testis poly A (+) RNA (Clontech cat # 6535-1) was first treated with DNAse I (Life Technologies) to destroy remaining traces of genomic DNA and then converted to cDNA via reverse transcription using the Superscript™ II reverse transcriptase (Life Technologies) according to the protocol recommended by the supplier of these enzymes. PCR primers were designed to amplify the putative IGS43 coding sequence. The primary PCR reaction (50 μl volume) was carried out on the testis cDNA template (originating from 50 ng DNAse I treated and reverse transcribed human polyA (+) testis RNA) using the HotStarTaq™ DNA polymerase (Qiagen # 203203) with forward and reverse primers IP14,923 (SEQ ID NO:3) and IP14,925 (SEQ ID NO:4) respectively, under the conditions recommended by Qiagen. For the PCR reaction, reaction tubes were heated at 95° C. for 15 min and then subjected to 35 cycles of denaturation (94° C., 30 sec), annealing (55° C., 30 sec) and extension (72° C., 90 sec). There was a final elongation for 10 min at 72° C. 2.5 μl of the primary PCR reaction was used as a template in a secondary PCR reaction using the Qiagen HotStarTaq™ DNA polymerase (Qiagen # 203203) with the nested forward and reverse primers IP14,924 (SEQ ID NO:5) and IP14,926 (SEQ ID NO:6) respectively. Cloning sites were added to the nested primers to allow convenient subcloning afterwards. Cycling conditions for the nested PCR reaction were identical to these of the primary PCR, except that only 30 cycles were carried out PCR reaction products were analysed via agarose gel electrophoresis and stained with ethidium bromide. The primary PCR reaction products showed up on gel as a smear of several bands. The nested PCR reaction however produced a strong distinct DNA fragment of approximately 1050 bp. No DNA products were obtained when mock reverse transcribed (no Superscript™ II enzyme added in the reverse transcription reaction) RNA was used, demonstrating that the 1050 bp fragment originated from cDNA. The +1050 bp fragment was purified from gel using the QIAEX™ II purification kit (Qiagen) and ligated into the pGEM-T vector according to the procedure recommended by the supplier (pGEM-T system, Promega). The recombinant plasmids were then used to transform competent DH5^{s}F′ bacteria. Transformed cells were plated on LB agar plates containing ampicillin (100 μg/ml), IPTG (0.5 mM) and X-gal (50 μg/ml). Plasmid DNA was purified from mini-cultures of individual white colonies using the BioRobot™ 9600 nucleic acid purification system (Qiagen) and sequenced. DNA sequencing reactions were carried out on the purified plasmid DNA with the ABI Prism™ BigDye™ Terminator Cycle Sequencing Ready Reaction kit (PE Applied Biosystems) using insert flanking and internal (IGS43 specific) primers. Cycle sequencing reaction products were purified via EtOH/NaOAc precipitation and loaded on an ABI 377 automated sequencer (PE Applied Biosystems). Clone HB6127 contained a DNA sequence of 1057 bp, encoding a protein of 327 amino acids. We refer to this DNA sequence and the encoded protein as IGS43DNA (SEQ ID NO:1) and IGS43PROT (SEQ ID NO:2) respectively. The IGS43DNA sequence was 97% identical to the stretch initially identified within the AC019124 genomic sequence (24 mismatches were found over 1057 aligned nucleotides). This is likely due to the fact that the sequence data of AC019124 were only of draft quality. For the IGS43PROT sequence homology searches of up to date protein databanks and translated DNA databanks were executed using the BLAST algorithm (Altschul S. F. et al. [1997], Nucleic Acids Res. 25: 3389-3402). These searches showed that the IGS43PROT sequence was most similar with the rat GPCR RTA (85% identities over 327 aligned residues; Swissprot accession n^{o} P23749). In the patent literature the application EP1067182 Seq Id 322 gives a protein for a probable G-protein coupled receptor. This protein is 16 amino acids longer, but otherwise identical to the IGS43 protein.

TABLE 3

Overview of the oligonucleotide primers that were
used to clone IGS43 cDNA.

| | | |
|---|---|---|
| SEQ ID NO:3 | IP14,923 | 5′-GCTCCACAGCCACTGCCTTACAGAC-3′ |
| SEQ ID NO:4 | IP14,925 | 5′-CACAGTCCCAGGGAGAAGAAGGC-3′ |
| SEQ ID NO:5 | IP14,924 | 5′-ACGGATTCATCACAGGTGTCCCTCTCTG |

C-3′

| | | |
|---|---|---|
| SEQ ID NO:6 | IP14,926 | 5′-AGAAGCTTGGAGCACGATTCCTGCCACT |

GC-3′
Cloning sites (intended BamHI restriction site for IP14,924 and HindIII restriction site for IP14,926) that were added to the nested primers are underlined.
Due to an error in the design of the BamHI restriction site within primer IP14,924 [“GGATTC” instead of “GGATCC”], this BamHI restriction site is not functional.

### EXAMPLE 2

### Construction of the Mammalian Expression Vector pcDNA3.1(−)hIGS43

The E. coli bacterial strain HB6127 harboring plasmid pGEM-ThsIGS43, which contained the complete coding sequence of the human IGS43 protein was recloned after replating on LB agar plates (containing 100 μg ampicillin/ml) and deposited both in Innogenetics' bacterial strain collection (ICCG 4555) and at the “Centraalbureau voor Schimmelcultures (CBS)” at Utrecht, The Netherlands (accession n^{o} 109714). Plasmid DNA prepared from the recloned isolate was resequenced and found to be identical to the IGS43DNA consensus sequence determined previously.

A ±1070 bp DNA fragment containing the complete IGS43 coding sequence, was PCR-amplified from the pGEM-ThsIGS43 plasmid template using oligonucleotide primers IP15,332 (SEQ ID NO:7) and IP15,333 (SEQ ID NO:8). At the 5′ end oligonucleotides IP15,332 (SEQ ID NO:7) and IP15,333 (SEQ ID NO:8) contained KpnI and XbaI cloning sites respectively. The amplified PCR fragment was Kpn/XbaI digested and purified from gel. The pcDNA3.1(+) plasmid expression vector (Invitrogen) was digested with Kpn/XbaI and the linearized 5362 bp vector fragment was purified from gel. The linearized plasmid vector and the PCR fragment were ligated and the ligation mixture was transformed into competent E. coli strain DH5αF′ bacteria by heat shock. Transformed bacteria were plated on LB agar plates (containing 100 μg/ml ampicilline) and incubated overnight at 37° C. Individual bacterial colonies were selected and cultured overnight in LB medium containing 100 μg/ml ampicillin. Plasmid DNA was prepared and analysed via DNA sequence analysis. One clone was deposited in Innogenetics' bacterial culture collection as ICCG 4694 (harboring plasmid pcDNA3.1(+)hIGS43).

DNA sequence analysis of plasmid DNA pcDNA3.1(+)hIGS43 prepared from strain ICCG 4694 showed that the insert was completely identical to the IGS43DNA consensus cDNA sequence.

### EXAMPLE 3

### Expression Analysis of IGS43 mRNA in Different Human Tissues Via Quantitative PCR (Q-PCR)

Absolute expression levels of human GAPDH (glyceraldehyde-3-phosphate dehydrogenase) and IGS43 mRNA were determined in a real-time quantitative RT-PCR assay (Q-PCR) using the Light Cycler™ Instrument (Roche Diagnostics) and gene specific PCR primers and TaqMan™ probes on human RNA samples. mRNA levels for the house keeping gene GAPDH were measured as a control for the efficiency of cDNA synthesis and PCR amplification on the different RNA samples.

cDNA was synthesized via reverse transcription from either total RNA of different human tissues (Clontech human RNA panels cat# K4000-1, K4001-1, K4002-1, K4003-1 and K4004-1) or from poly(A)⁺ RNA derived from different subregions of human brain (Clontech cat #6580.1, 6575.1, 6543.1, 6574.1, 6577.1, 6578.1 and 6582.1). Prior to reverse transcription RNA was treated with DNAse I (Life Technologies cat# 18068-015) according to the procedure recommended by the supplier in order to destroy possible contaminating genomic DNA. The DNAse I reaction was stopped by adding EDTA (final concentration=2.3 mM) and heating for 10 min at 65° C. DNAse I treated RNA (either 5 μg total RNA or 945 ng poly(A)⁺ RNA) was annealed to 2.5 μg oligo(dT) (Life Technologies # 18418-012) and subjected to reverse transcription using Omniscript Reverse Transcriptase (Qiagen; 100 μl reaction volume; 1 h at 37° C.) (=RT⁺-reaction). The reverse transcriptase was inactivated by incubation at 93° C. for 5 min. Part of the DNAse treated RNA was not subjected to reverse transcription and was used as a control to check for the presence of remaining genomic DNA (RT⁻-reaction).

As a control for the absence of genomic DNA in the RT⁻ reactions of the total RNA samples a PCR amplification reaction specific for human β₂-microglobulin DNA was performed. The PCR reaction was carried out in a 25 μl reaction volume containing 2.5 μl GeneAmp™ 10×PCR buffer (Applied Biosystems), 200 μM each of dNTP, 0.5 μl of the RT⁻ cDNA synthesis reaction, 5 pmol each of PCR primers IP3,981 (SEQ ID NO:9) and IP3,982 (SEQ ID NO:10) and 1.25 U AmpliTaq Gold™ DNA polymerase (Applied Biosystems cat# N808-0244). After an initial incubation at 95° C. for 10 min, reactions were cycled 30 times as follows: 1 min denaturation at 94° C., 1 min annealing at 55° C. and 1 min extension at 72° C. There was a final extension at 72° C. for 10 min. As a positive control 50 ng of human genomic DNA (Clontech #6551-1) was used. An amplicon of expected length was obtained from the genomic DNA template but not from the negative control (H₂O) nor from the RT⁻ samples (due to the fact that the IP3,981/IP3982 primer pair spans a 616 bp intron the predicted amplicon lengths are 269 bp and 885 bp on cDNA and genomic DNA respectively). RT⁻ poly(A)⁺ RNA samples were analyzed for the presence of genomic DNA via GAPDH specific Q-PCR using 0.8 μl of the RT cDNA synthesis reaction (see below). No GAPDH specific signal was obtained. We concluded that the synthesized cDNA was free of genomic DNA.

Q-PCR reactions were carried out in a 20 μl reaction volume containing 1×TaqMan™ Universal PCR Master Mix (Applied Biosystems cat# 4304437), 0.12 mg BSA/ml, 900 nM of either GAPDH or IGS43 specific forward and reverse primers (IP15,529 [SEQ ID NO:11]/IP15,531 [SEQ ID NO:12] for GAPDH and IP17,080 [SEQ ID NO:13]/IP 17,081 [SEQ ID NO:14] for IGS43), 250 nM of the gene specific TaqMan probe (IP15,530 [SEQ ID NO:15] for GAPDH and IP 17,082 [SEQ ID NO:16] for IGS43 respectively) and 1.6 μl of the total RNA RT⁺ or poly(A)⁺ RT⁺ cDNA synthesis reaction. The 1×TaqMan™ Universal PCR Master Mix contained AmpliTaq Gold™ DNA polymerase, AmpErase™ Uracil N-glycosylase (UNG), dNTPs with dUTP, Passive Reference 1 and optimized buffer components. Specific primers and TaqMan probes were designed with the Primer Express™ software (Applied Biosystems). Gene specific standard curves were established on a 1/10 dilution series (10⁸ to 10² copies/reaction) of linearized pGEM-ThuGAPDH (containing full length human GAPDH cDNA) or pcDNA3.1(+)hIGS43 plasmid (ICCG 4694). PCR reactions were carried out in glass capillary cuvettes in the Light Cycler™ instrument. After an initial incubation at 50° C. for 2 min (to allow the AmpErase UNG reaction), followed by the activation of the AmpliTaq Gold™ DNA polymerase for 10 min at 95° C., reactions were cycled 50 times as follows: 15 sec denaturation at 95° C. and 60 sec annealing/extension at 60° C. (for both GAPDH and IGS43). Quantification of the samples was carried out using the Light Cycler Software version 3.0.

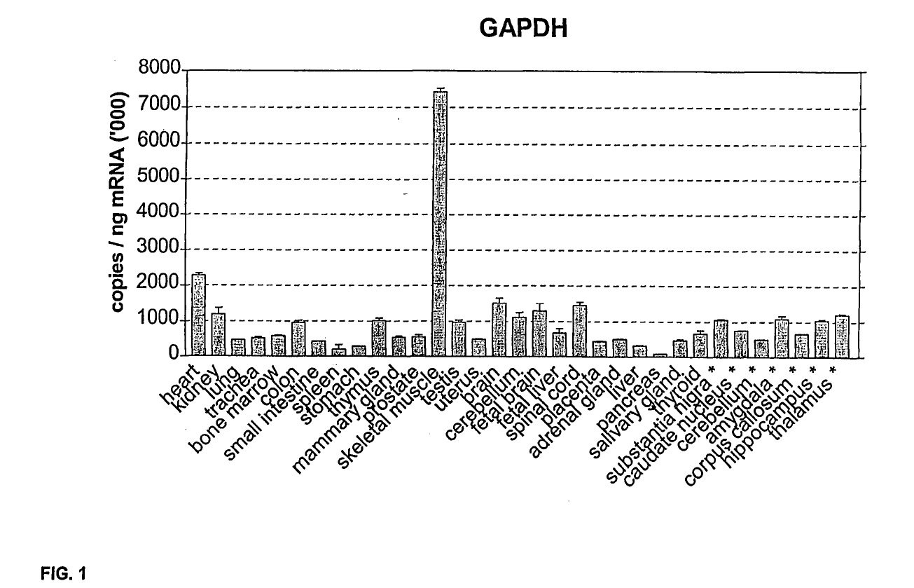

Absolute expression levels for human GAPDH mRNA ranged from ≈4×10⁵ to ≈1.5×10⁶ copies/ng poly(A)⁺ RNA in most tissues except in skeletal muscle (≈7.4×10⁶ copies/ng poly(A)⁺ RNA), heart (≈2.3×10⁶ copies/ng poly(A)⁺ RNA) and in pancreas, spleen, liver and stomach (ranging between ≈1-3×10⁵ copies/ng poly(A)⁺ RNA respectively) (FIG. 1).

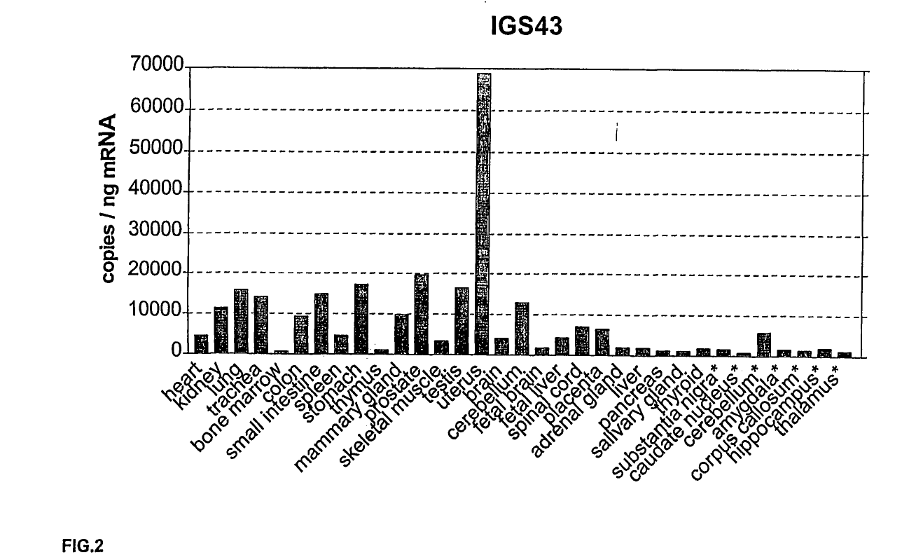

IGS43 mRNA was found to be most abundantly expressed in uterus (±69,000 copies/ng mRNA) (FIG. 2). Important levels (between approximately 10,000 and 20,000 copies/ng mRNA) were also found in kidney, lung, trachea, colon, small intestine, stomach, mammary gland, prostate and testis. Relatively minor levels were found in the central nervous system apart from cerebellum and spinal cord.

TABLE 4

Overview of the oligonucleotide primers and
Taqman probes that were used.

| | | |
|---|---|---|
| SEQ ID NO:7 | IP15,332 | 5′-GGGGTACCATCACAGGTGTCCCTCTCTGC-3′ |
| SEQ ID NO:8 | IP15,333 | 5′-GCTCTAGAGCACGATTCCTGCCACTGCC-3′ |
| SEQ ID NO:9 | IP3,981 | 5′-CCAGCAGAGAATGGAAAGTC-3′ |
| SEQ ID NO:10 | IP3,982 | 5′-GATGCTGCTTACATGTCTCG-3′ |
| SEQ ID NO:11 | IP15,529 | 5′-GGTGAAGCAGGCGTCGG-3′ |
| SEQ ID NO:12 | IP15,531 | 5′-GACAAAGTGGTCGTTGAGGGC-3′ |
| SEQ ID NO:13 | IP17,080 | 5′-CCTGGCCATGGTCTCCG-3′ |
| SEQ ID NO:14 | IP17,081 | 5′-ACGATGGGCTTGGCGC-3′ |
| SEQ ID NO:15 | IP15,530 | 5′(FAM)-TGGTCTCCTCTGACTTCAACAGCGACACC- |
| | TaqMan probe | (TAMRA)3′ |
| SEQ ID NO:16 | IP 17,082 | 5′(FAM)-CCGGCCCCCTTCCCCGAGT-(TAMRA)3′ |
| | TaqMan probe | |

### EXAMPLE 4

### Construction of IGS43 Transfected Cells

To identify ligands for IGS43, Chinese Hamster Ovary (CHO) cells are stably transfected with cDNA of the IGS43 orphan receptor. Since the G-protein coupling mechanism of IGS43 receptor is still unknown, a specific CHO-cell strain is used, which expresses the G-protein Gα16 (CHO-K1-Gα16, Molecular Devices), known as “universal adapter” for GPCRs (Milligan G. et al. (1996) Trends Pharmacol. Sci. 17: 235-7). This cell line also stably expresses the mitochondrially targeted apo-aequorin.

The Materials include: IGS43-pcDNA3.1 vector; SuperFect Transfection Reagent (Qiagen); Growth-medium: CHO-S-SFM II (Gibco BRL), supplemented with 10% Foetal Calf Serum (FCS, Gibco BRL), 2 mM L-glutamin, Hygromycin B 400 μg/ml; Selection-medium: CHO-S-SFM II (Gibco BRL), supplemented with 10% FCS, 2 mM L-glutamin, Hygromycin B 400 μg/ml and Geneticin 500 μg/ml; RNeasy Mini Kit (Qiagen), DNase I (Ambion, 2 U/μl), SuperScript II (Gibco BRL), SuperScript II 200U (Gibco BRL).

CHO-K1-Gα16/mtAEQ cells are transfected with SuperFect (Qiagen), as described by the manufacturer. Transfections are done in a 24 wells plate. After 24 hours in Growth-medium, medium is removed and replaced by Selection-medium. After growing to confluency in Selection-medium the polyclonals are passed once in a 24 wells plate.

Selection of polyclonals is done by Q-PCR RNA is isolated from monoclonals (1 confluent well from 24 wells plate) with the RNeasy Mini Kit (Qiagen), according to the supplied protocol. RNA is treated with DNase I (Ambion, 2 U/μl), 1 U per sample. Half of the RNA sample is used for RT-PCR using SuperScript II (Gibco BRL). Primer annealing is carried out with RNA and oligo-dT16 (0.6 μM) for 10 min at 65° C. to 15° C. First Strand Buffer (Gibco BRL) with dNTP's 0.43 mM each, DTT 10 mM, 20U RNasin (Promega, 40 U/μl) and SuperScript II 200U (Gibco BRL, 200 U/μl) to a final volume of 30 μl are added, followed by incubation at 42° C. for 1 hour.

Q-PCR is carried out with IGS43 receptor specific Q-PCR primers. The amount of PCR product is determined after each cycle by measuring the fluorescence of Sybr Green, which binds to dsDNA. The relative expression level of the IGS is related to a standard curve of four different dilutions of chromosomal DNA. The relative quantification is normalized against the housekeeping gene Beta-Tubulin.

The two best polyclonals are used to obtain monoclonals. Cells are seeded in Limited Dilution. Selection of monoclonals is done by Q-PCR, as described earlier. The six best monoclonals are grown in T75 flask to confluency and frozen in growth medium, containing 10% DMSO.

### EXAMPLE 5

### Ligand Finding

CHO-K1-Gα16-mtAEQ cells expressing the particular G-protein coupled receptor are grown as described in Example 4 and used in screening a number of compound libraries. Compounds are tested at a concentration of 1 or 10 μM. In the aequorin screening assay ATP (10 μM) or digitonin (50 μM) is used as a positive control. Screening is performed semi-automatically using a MicroBeta Jet 1450 (Perkin Elmer) as described below.

Once compounds are found showing a signal activity is confirmed in a second aequorin-experiment. Subsequently they are characterized further in dose-response experiments.

If not successful in finding ligands using CHO-K1-Gα16 cells expressing IGS43 receptor and apo-aequorin a corresponding cell line without G-protein Gal 6 can be developed in a similar way (see Example 4) followed by testing compounds.

[image]

[image]

[image]

1. An isolated polynucleotide selected from the group consisting of:
a) a nucleotide sequence encoding the IGS43 polypeptide according to SEQ ID NO: 2;
b) a nucleotide sequence encoding the polypeptide encoded by the DNA insert contained in the deposit no. CBS 109714 at the Centraalbureau voor Schimmelcultures at Utrecht (The Netherlands), in particular a nucleotide sequence corresponding to the SEQ ID NO: 1;
c) a nucleotide sequence having at least 95.5% (preferably at least 96%) sequence identity over its entire length to the nucleotide sequence of (a) or (b);
d) a nucleotide sequence which is complimentary to the nucleotide sequence of (a) or (b) or (c).

2. An isolated polynucleotide selected from the group consisting of:
a) a nucleotide sequence encoding the IGS43 polypeptide according to SEQ ID NO: 2;
b) a nucleotide sequence encoding the polypeptide encoded by the DNA insert contained in the deposit no. CBS 109714 at the Centraalbureau voor Schimmelcultures at Utrecht (The Netherlands), in particular a nucleotide sequence corresponding to the SEQ ID NO: 1;
c) a nucleotide sequence having at least 96% (preferably at least 97%) sequence identity over its entire length to the nucleotide sequence of (a) or (b);
d) a nucleotide sequence which is complimentary to the nucleotide sequence of (a) or (b) or (c).

3. An isolated polynucleotide selected from the group consisting of:
a) a nucleotide sequence encoding the IGS43 polypeptide according to SEQ ID NO: 2;
b) a nucleotide sequence encoding the polypeptide encoded by the DNA insert contained in the deposit no. CBS 109714 at the Centraalbureau voor Schimmelcultures at Utrecht (The Netherlands), in particular a nucleotide sequence corresponding to the SEQ ID NO: 1;
c) a nucleotide sequence having at least 97% (preferably at least 98%) sequence identity over its entire length to the nucleotide sequence of (a) or (b);
d) a nucleotide sequence which is complimentary to the nucleotide sequence of (a) or (b) or (c).

4. An isolated polynucleotide selected from the group consisting of:
a) a nucleotide sequence encoding the IGS43 polypeptide according to SEQ ID NO: 2;
b) a nucleotide sequence encoding the polypeptide encoded by the DNA insert contained in the deposit no. CBS 109714 at the Centraalbureau voor Schimmelcultures at Utrecht (The Netherlands), in particular a nucleotide sequence corresponding to the SEQ ID NO: 1;
c) a nucleotide sequence having at least 98% (preferably at least 99%) sequence identity over its entire length to the nucleotide sequence of (a) or (b);
d) a nucleotide sequence which is complimentary to the nucleotide sequence of (a) or (b) or (c).

5. The polynucleotide of any one of claims 1 to 4 wherein said polynucleotide consists of the nucleotide sequence contained in SEQ ID NO:1 encoding the IGS43 polypeptide of SEQ ID NO:2.

6. The polynucleotide of any one of claims 1 to 4 wherein said polynucleotide consists of a nucleotide sequence that is at least 95.5% identical to that of SEQ ID NO:1 or to the sequence of the DNA insert contained in the deposit no. CBS 109714 at the Centraalbureau voor Schimmelcultures at Utrecht (The Netherlands) over its entire length.

7. The polynucleotide of any one of claims 1 to 4 wherein said polynucleotide consists of a nucleotide sequence that is at least 96% identical to that of SEQ ID NO:1 or to the sequence of the DNA insert contained in the deposit no. CBS 109714 at the Centraalbureau voor Schimmelcultures at Utrecht (The Netherlands) over its entire length.

8. The polynucleotide of any one of claims 1 to 4 wherein said polynucleotide consists of a nucleotide sequence that is at least 97% identical to that of SEQ ID NO:1 or to the sequence of the DNA insert contained in the deposit no. CBS 109714 at the Centraalbureau voor Schimmelcultures at Utrecht (The Netherlands) over its entire length.

9. The polynucleotide of any one of claims 1 to 4 wherein said polynucleotide consists of a nucleotide sequence that is at least 98% identical to that of SEQ ID NO:1 or to the sequence of the DNA insert contained in the deposit no. CBS 109714 at the Centraalbureau voor Schimmelcultures at Utrecht (The Netherlands) over its entire length.

10. The polynucleotide of any one of claims 1 to 4 wherein said polynucleotide consists of a nucleotide sequence that is at least 99% identical to that of SEQ ID NO:1 or to the sequence of the DNA insert contained in the deposit no. CBS 109714 at the Centraalbureau voor Schimmelcultures at Utrecht (The Netherlands) over its entire length.

11. The polynucleotide of any one of claims 5 to 10 which is the polynucleotide of SEQ ID NO:1 or the DNA insert contained in the deposit no. CBS 109714 at the Centraalbureau voor Schimmelcultures at Utrecht (The Netherlands).

12. The polynucleotide of any one of claims 1 to 11 which is DNA or RNA.

13. A hybridization probe consisting of the polynucleotide of any one of claims 1 to 4.

14. A DNA or RNA molecule comprising an expression system, wherein said expression system is capable of producing an IGS43 polypeptide consisting of an amino acid sequence, which has at least 95.5% identity with the polypeptide of SEQ ID NO:2 or with the polypeptide encoded by the DNA insert contained in the deposit no. CBS 109714 at the Centraalbureau voor Schimmelcultures at Utrecht (The Netherlands), when said expression system is present in a compatible host cell.

15. A DNA or RNA molecule comprising an expression system, wherein said expression system is capable of producing an IGS43 polypeptide consisting of an amino acid sequence, which has at least 96% identity with the polypeptide of SEQ ID NO:2 or with the polypeptide encoded by the DNA insert contained in the deposit no. CBS 109714 at the Centraalbureau voor Schimmelcultures at Utrecht (The Netherlands), when said expression system is present in a compatible host cell.

16. A DNA or RNA molecule comprising an expression system, wherein said expression system is capable of producing an IGS43 polypeptide consisting of an amino acid sequence, which has at least 97% identity with the polypeptide of SEQ ID NO:2 or with the polypeptide encoded by the DNA insert contained in the deposit no. CBS 109714 at the Centraalbureau voor Schimmelcultures at Utrecht (The Netherlands), when said expression system is present in a compatible host cell.

17. A DNA or RNA molecule comprising an expression system, wherein said expression system is capable of producing an IGS43 polypeptide consisting of an amino acid sequence, which has at least 98% identity with the polypeptide of SEQ ID NO:2 or with the polypeptide encoded by the DNA insert contained in the deposit no. CBS 109714 at the Centraalbureau voor Schimmelcultures at Utrecht (The Netherlands), when said expression system is present in a compatible host cell.

18. A DNA or RNA molecule comprising an expression system, wherein said expression system is capable of producing an IGS43 polypeptide consisting of an amino acid sequence, which has at least 99% identity with the polypeptide of SEQ ID NO:2 or with the polypeptide encoded by the DNA insert contained in the deposit no. CBS 109714 at the Centraalbureau voor Schimmelcultures at Utrecht (The Netherlands), when, said expression system is present in a compatible host cell.

19. A host cell comprising the expression system of any one of claims 14 to 18.

20. A host cell according to claim 19 which is a yeast cell

21. A host cell according to claim 19 which is an animal cell

22. IGS43 receptor membrane preparation derived from a cell according to any one of claims 19 to 21.

23. A process for producing an IGS43 polypeptide comprising culturing a host of claim 19 to 21 under conditions sufficient for the production of said polypeptide and recovering the polypeptide from the culture.

24. A process for producing a cell which produces an IGS43 polypeptide thereof comprising transforming or transfecting a cell with the expression system of any one of claims 14 to 18 such that the cell, under appropriate culture conditions, is capable of producing an IGS43 polypeptide.

25. An IGS43 polypeptide consisting of an amino acid sequence which is at least 95.5% identical to the amino acid sequence of SEQ ID NO:2 or to the polypeptide encoded by the DNA insert contained in the deposit no. CBS 109714 at the Centraalbureau voor Schimmelcultures at Utrecht (The Netherlands) over its entire length.

26. An IGS43 polypeptide consisting of an amino acid sequence which is at least 96% identical to the amino acid sequence of SEQ ID NO:2 or to the polypeptide encoded by the DNA insert contained in the deposit no. CBS 109714 at the Centraalbureau voor Schimmelcultures at Utrecht (The Netherlands) over its entire length.

27. An IGS43 polypeptide consisting of an amino acid sequence which is at least 97% identical to the amino acid sequence of SEQ ID NO:2 or to the polypeptide encoded by the DNA insert contained in the deposit no. CBS 109714 at the Centraalbureau voor Schimmelcultures at Utrecht (The Netherlands) over its entire length.

28. An IGS43 polypeptide consisting of an amino acid sequence which is at least 98% identical to the amino acid sequence of SEQ ID NO:2 or to the polypeptide encoded by the DNA insert contained in the deposit no. CBS 109714 at the Centraalbureau voor Schimmelcultures at Utrecht (The Netherlands) over its entire length.

29. An IGS43 polypeptide consisting of an amino acid sequence which is at least 99% identical to the amino acid sequence of SEQ ID NO:2 or to the polypeptide encoded by the DNA insert contained in the deposit no. CBS 109714 at the Centraalbureau voor Schimmelcultures at Utrecht (The Netherlands) over its entire length.

30. The polypeptide of any one of claims 25 to 29 which consists of the amino acid sequence of SEQ ID NO:2 or the amino acid sequence encoded by the DNA insert contained in the deposit no. CBS 109714 at the Centraalbureau voor Schimmelcultures at Utrecht (The Netherlands).

31. An antibody immunospecific for the IGS43 polypeptide of any one of claims 25 to 30.

32. A method for the treatment of a subject in need of enhanced activity or expression of IGS43 polypeptide receptor of any one of claims 25 to 30 comprising:
(a) administering to the subject a therapeutically effective amount of an agonist to said receptor; and/or
(b) providing to the subject an isolated polynucleotide consisting of a nucleotide sequence that has at least 95.5% identity to a nucleotide sequence encoding the IGS43 polypeptide of SEQ ID NO:2 or the polypeptide encoded by the DNA insert contained in the deposit no. CBS 109714 at the Centraalbureau voor Schimmelcultures at Utrecht (The Netherlands) over its entire length; or a nucleotide sequence complementary to said nucleotide sequence in a form so as to effect production of said receptor activity in vivo.

33. A method for the treatment of a subject having need to inhibit activity or expression of IGS43 polypeptide receptor of any one of claims 25 to 30 comprising:
(a) administering to the subject a therapeutically effective amount of an antagonist to said receptor; and/or
(b) administering to the subject a polynucleotide that inhibits the expression of the nucleotide sequence encoding said receptor, and/or
(c) administering to the subject a therapeutically effective amount of a polypeptide that competes with said receptor for its ligand.

34. A process for diagnosing a disease or a susceptibility to a disease in a subject related to expression or activity of the IGS43 polypeptide of any one of claims 25 to 30 in a subject comprising:
(a) determining the presence or absence of a mutation in the nucleotide sequence encoding said IGS43 polypeptide in the genome of said subject in a sample derived from said subject; and/or
(b) analyzing for the presence or amount of the IGS43 polypeptide expression in a sample derived from said subject.

35. A method for identifying agonists to the IGS43 polypeptide of any one of claims 25 to 30 comprising:
(a) contacting a cell which produces a IGS43 polypeptide with a test compound; and
(b) determining whether the test compound effects a signal generated by activation of the IGS43 polypeptide.

36. An agonist identified by the method of claim 35.

37. A method for identifying antagonists to the IGS43 polypeptide of any one of claims 25 to 30 comprising:
(a) contacting a cell which produces a IGS43 polypeptide with an agonist; and
(b) determining whether the signal generated by said agonist is diminished in the presence of a candidate compound.

38. An antagonist identified by the method of claim 37.

39. A recombinant host cell produced by a method of claim 24 or a membrane thereof expressing an IGS43 polypeptide.

40. A method of creating a genetically modified non-human animal comprising the steps of:
a) ligating the coding portion of a polynucleotide consisting of a nucleic acid sequence encoding a protein having the amino acid sequence SEQ ID NO: 2 or the amino acid sequence encoded by the DNA insert contained in the deposit no. CBS 109714 at the Centraalbureau voor Schimmelcultures at Utrecht (The Netherlands) to a regulatory sequence which is capable of driving high level gene expression or expression in a cell type in which the gene is not normally expressed in said animal; or
b) engineering the coding portion of a polynucleotide consisting of a nucleic acid sequence encoding a protein having the amino acid sequence SEQ ID NO: 2 or the amino acid sequence encoded by the DNA insert contained in the deposit no. CBS 109714 at the Centraalbureau voor Schimmelcultures at Utrecht (The Netherlands), and reintroducing said sequence in the genome of said animal in such a way that the endogenous gene alleles, encoding a protein having the amino acid sequence SEQ ID NO: 2 or the amino acid sequence encoded by the DNA insert contained in the deposit no. CBS 109714 at the Centraalbureau voor Schimmelcultures at Utrecht (The Netherlands), are fully or partially inactivated.

41. A method for the production of a pharmaceutical composition comprising the method of claim 35 or 37 and then mixing the compound identified with a pharmaceutically acceptable carrier.

42. Use of:
(a) a therapeutically effective amount of an agonist to the IGS43 receptor polypeptide of any one of claims 25 to 30; and/or
(b) an isolated polynucleotide consisting of a nucleotide sequence that has at least 95.5% identity to a nucleotide sequence encoding the IGS43 polypeptide of SEQ ID NO: 2 or the polypeptide encoded by the DNA insert contained in the deposit no. CBS 109714 at the Centraalbureau voor Schimmelcultures at Utrecht (the Netherlands) over its entire length; or a nucleotide sequence complementary to said nucleotide sequence in a form so as to effect production of said receptor activity in vivo,
for the preparation of a medicament for the treatment of a subject suffering from a disease related to expression or activity of the IGS43 receptor polypeptide, in need of enhanced activity or expression of IGS43 polypeptide of any one of claims 25 to 30.

43. Use of
(a) a therapeutically effective amount of an antagonist to the IGS43 receptor polypeptide of any one of claims 25 to 30; and/or
(b) a nucleic acid molecule that inhibits the expression of the nucleotide sequence encoding the IGS43 receptor polypeptide of any one of claims 25 to 30; and/or
(c) a therapeutically effective amount of a polypeptide that competes with the IGS43 receptor polypeptide of any one of claims 25 to 30 for its ligand,
for the preparation of a medicament for the treatment of a subject suffering from a disease related to expression or activity of the IGS43 receptor polypeptide, having need to inhibit activity or expression of IGS43 polypeptide of any one of claims 25 to 30.
